(19) United States
(12) Patent Application Publication
(10) Pub. No.: US 2026/0202410 A1
(43) Pub. Date: Jul. 16, 2026

(54) Assay for Detecting Cancer by Using an Antibody that Binds to C-Terminal Epitope of Type IX Collagen

(71) Applicant: Nordic Bioscience A/S, Herlev (DK)

(72) Inventors: Helena Port Linares, København N (DK); Signe Holm Nielsen, Frederiksberg (DK); Anne-Christine Bay-Jensen, København S (DK); Morten Karsdal, København Ø (DK)

(73) Assignee: Nordic Bioscience A/S, Herlev (DK)

(21) Appl. No.: 19/134,063

(22) PCT Filed: Nov. 29, 2023
(86) PCT No.: PCT/EP2023/083529

§ 371 Date: May 29, 2025

(30) Foreign Application Priority Data
Nov. 30, 2022 (GB) 2218042.6

(51) Int. Cl. G01N 33/5752; C07K 16/18

(52) U.S. Cl. CPC G01N 33/5752; C07K 16/18; C07K 2317/565; C07K 2317/92; C07K 2317/94; G01N 2333/78; G01N 2470/10

(57) ABSTRACT

The present invention relates to methods of immunoassay suitable for detecting and/or monitoring a cancer in a patient, immunoassay kits suitable for carrying out the methods and antibodies suitable for use in the methods and kits.

### FIELD OF INVENTION

The present invention relates to methods of immunoassay suitable for detecting and/or monitoring a cancer in a patient, immunoassay kits suitable for carrying out said methods and antibodies suitable for use in said methods and kits.

### BACKGROUND

Lung cancer is the most diagnosed cancer and the leading cause of cancer death worldwide(1). Non-small cell lung cancer (NSCLC) represents approximately 85% of all lung cancer cases, wherein adenocarcinoma and squamous cell carcinoma are the most common subtypes(2). One of the major components of the tumor microenvironment is the extracellular matrix (ECM), the non-cellular part of tissues(3). Collagens are the most prominent ECM proteins, of which there are 28 different types with 46 sidechains(4).

It has previously been shown that fragments of fibril-associated collagen with interrupted triple helices (FACIT) collagens are associated with cancer(5). Localization and a thorough description of type IX collagen in human lungs is lacking in the literature. It has been extensively studied in human articular cartilage, where it is colocalized with type II collagen(6,7). A previous study by Chung et al from 2017 indicated that certain single nucleotide polymorphisms (SNPs) in the COL9A1 gene are associated with an increased risk of oral cancer(8). Piotrowski et al 2006 demonstrated by microarray hybridisation and bisulphite sequencing a reduction in methylation of CpG islands in the COL9A1 gene in breast cancer tumour tissue samples(9).

### SUMMARY

The applicant has now developed a method of immunoassay targeting the C-terminus amino acid sequence QRAFNKGPDP (referred to herein as “PRO-C9”, “target sequence” or “PRO-C9 target sequence”) of the type IX collagen α-1 chain, and has demonstrated that elevated levels of PRO-C9 are present in and can be detected in serum samples from patients with a number of different cancers including non-small cell lung cancer (NSCLC), thereby demonstrating the utility of said method of immunoassay as a means for detecting and/or monitoring cancer.

Accordingly, in a first aspect the present invention provides a method of immunoassay, the method comprising;
i) contacting a patient sample with a monoclonal antibody that specifically binds to the C-terminus amino acid sequence QRAFNKGPDP (SEQ ID No. 1);
ii) detecting and determining the amount of binding between said monoclonal antibody and peptides in the sample.

Preferably, the method is a method of immunoassay for detecting and/or monitoring a cancer in a patient, the method further comprising;
iii) correlating said amount of binding with values associated with normal healthy subjects and/or values associated with known disease severity and/or values obtained from said patient at a previous time point and/or with a predetermined cut-off value.

As used herein the term “C-terminus” refers to a C-terminal peptide sequence at the extremity of a polypeptide, i.e. at the C-terminal end of the polypeptide, and is not to be construed as meaning in the general direction thereof.

As used herein, the terms “peptide” and “polypeptide” are used synonymously.

A monoclonal antibody suitable for use in the method of the invention is disclosed herein which specifically binds to an epitope consisting of or within the C-terminal amino acid sequence QRAFNKGPDP-COOH (SEQ ID NO:1).

Preferably, the monoclonal antibody does not substantially recognise or bind an elongated version of said C-terminal amino acid sequence which is QRAFNKGPDPG—COOH (SEQ ID NO: 2), i.e. a version of the PRO-C9 target sequence extended at the C-terminus by the addition of a glycine residue.

Preferably, the monoclonal antibody does not recognise or bind a shortened version of said C-terminal amino acid sequence having the amino acid sequence QRAFNKGPD-COOH (SEQ ID NO: 3).

As used herein the term “monoclonal antibody” refers to both whole antibodies and to fragments thereof that retain the binding specificity of the whole antibody, such as for example a Fab fragment, F(ab′)2 fragment, single chain Fv fragment, nanobodies, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the present invention, a monoclonal antibody comprising any constant region known in the art can be used. In the case of mouse antibodies and human antibodies, the constant light chains are classified as either kappa or lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgG1, IgG2, IgG3, and IgG4 in the case of humans and IgG1, IgG2a, IgG2b, IgG2c and IgG3 in the case of mice. The monoclonal antibody may preferably be of the IgG isotype, including any one of the IgG subclasses.

The CDR of an antibody can be determined using methods known in the art such as that described by Kabat et al. Antibodies can be generated from B cell clones. The isotype of the antibody can be determined by ELISA specific for IgM, IgG or IgA isotype, or subclass. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example, primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

Monoclonal antibodies that specifically bind to the C-terminus amino acid sequence QRAFNKGPDP—COOH (SEQ ID No. 1) can be generated via any suitable techniques known in the art. For example, the monoclonal antibody may be raised against a synthetic peptide having the amino acid sequence QRAFNKGPDP (SEQ ID No. 1), such as for example by: immunizing a rodent (or other suitable mammal) with a synthetic peptide consisting of the sequence QRAFNKGPDP (SEQ ID No. 1), which optionally may linked to an immunogenic carrier protein (such as keyhole limpet hemocyanin), isolating and cloning a single antibody producing cell, and assaying the resulting monoclonal antibodies to ensure that they have the desired specificity. An exemplary protocol for producing a monoclonal antibody that that specifically binds to the C-terminus amino acid sequence QRAFNKGPDP (SEQ ID No. 1) is described infra.

Preferably, the monoclonal antibody or fragment thereof may comprise one or more complementarity-determining regions (CDRs) selected from:

CDR-L1:
(SEQ ID No. 4)
KSSQSLLYSSNOMNYLA
CDR-L2:
(SEQ ID No. 5)
WASTRES
CDR-L3:
(SEQ ID No. 6)
HQYFSSRT
CDR-H1:
(SEQ ID No. 7)
IYTMN
CDR-H2:
(SEQ ID No. 8)
RIRSKSENYATYYADSVKD
CDR-H3:
(SEQ ID No. 9)
QGIYYDYYGAMDY

Preferably the antibody or fragment thereof comprises at least 2, 3, 4, 5 or 6 of the above listed CDR sequences.

Preferably the monoclonal antibody or fragment thereof has a light chain variable region comprising the CDR sequences

CDR-L1:
(SEQ ID No. 4)
KSSQSLLYSSNOMNYLA
CDR-L2:
(SEQ ID No. 5)
WASTRES
CDR-L3:
(SEQ ID No. 6)
HQYFSSRT

Preferably the monoclonal antibody or fragment thereof has a light chain that comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID NO. 10)
KSSQSLLYSSNOMNYLAWYQQKPGQSPKLLIYWASTRESDVPDRFTGSGSG
TDFTLTISSVQTEDLAVYYCHQYFSSRT

Preferably the monoclonal antibody or fragment thereof has a heavy chain variable region comprising the CDR sequences

CDR-H1:
(SEQ ID No. 7)
IYTMN
CDR-H2:
(SEQ ID No. 8)
RIRSKSENYATYYADSVKD
CDR-H3:
(SEQ ID No. 9)
QGIYYDYYGAMDY

Preferably the monoclonal antibody or fragment thereof has a heavy chain that comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID NO. 11)
IYTMNWVRQAPGKGLEWVARIRSKSENYATYYADSVKDRFTISRDDSQSIL
YLQMNNLKTEDTAIYYCVRQGIYYDYYGAMDY

As used herein, the framework amino acid sequences between the CDRs of an antibody are substantially identical or substantially similar to the framework amino acid sequences between the CDRs of another antibody if they have at least 70%, 80%, 90% or at least 95% similarity or identity. The similarity or identity may be measured over the entire length of each intervening framework sequence. The similar or identical amino acids may be contiguous or non-contiguous.

The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp. A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In certain preferred embodiments, the monoclonal antibody or fragment thereof may comprise the light chain variable region sequence:

(SEQ ID No. 12)
NIMMTQSPSSLAVSAGEKATMSCKSSQSLLYSSNOMNYLAWYQQKPGQSPK
LLIYWASTRESDVPDRFTGSGSGTDFTLTISSVQTEDLAVYYCHQYFSSRT
FGGGTKLEIK
(CDRs bold and underlined; Framework sequences in
italics)

and/or the heavy chain variable region sequence:

(SEQ ID No. 13)
EVQLVESGGGLVQPKGSLKLSCAASGFTFNIYTMNWVRQAPGKGLEWVARI
RSKSENYATYYADSVKDRFTISRDDSQSILYLOMNNLKTEDTAIYYCVRQG
IYYDYYGAMDYWGQGTSVTVS
(CDRs bold and underlined; Framework sequences in
italics)

As used herein the term “amount of binding” refers to the quantification of binding between the antibody and peptides in the patient sample. Said quantification may for example be determined by comparing the measured values of binding in the patient sample against a calibration curve produced using measured values of binding in standard samples containing known concentrations of a peptide to which the antibody specifically binds, in order to determine the quantity of peptide to which the antibody specifically binds in the patient sample. In the Examples set out below, an ELISA method is used in which spectrophotometric analysis is used to measure the amount of binding both in the patient samples and when producing the calibration curve. However, any suitable analytical method can be used.

The term “specifically bind” as used herein means that the antibody binding is selective for the antigen and that this binding can be distinguished from unwanted or non-specific interactions. The ability of a monoclonal antibody to bind to a specific epitope or peptide sequence can be measured either through an enzyme-linked immunosorbent assay (ELISA) as described herein or other techniques familiar to one of skill in the art, e.g. surface plasmon resonance (SPR) technique (analyzed e.g. on a BIAcore instrument) and traditional binding assays. The extent of binding of a monoclonal antibody to an unrelated protein is less than about 10% of the binding of the monoclonal antibody to the epitope or peptide as measured, e.g., by ELISA. “Affinity” refers to the strength of the sum total of non-covalent interactions between a single binding site of a molecule (e.g., an epitope binding region of an antibody) and its binding partner (e.g., an epitope or antigen). Unless indicated otherwise, as used herein, “binding affinity” refers to intrinsic binding affinity which reflects a 1:1 interaction between members of a binding pair (e.g., an antigen binding moiety and an antigen). The affinity of a molecule for its partner can generally be represented by the dissociation constant (Kd), which is the ratio of dissociation and association rate constants (koff and kon, respectively). Thus, equivalent affinities may comprise different rate constants, as long as the ratio of the rate constants remains the same. The dissociation constant represents the concentration of the antigen at which half of the binding sites on the antibody are occupied. A lower Kd indicates a higher binding affinity between the antibody and antigen, while a higher Kd reflects weaker binding. Several methods are available to measure the Kd of an antibody, including surface plasmon resonance (SPR), isothermal titration calorimetry (ITC), and fluorescence-based assays. In certain aspects, a monoclonal antibody that binds to the epitope or peptide has a dissociation constant (KD) of <1 pM, <100 nM, <10 nM, <1 nM, <0.1 nM, <0.01 nM, or <0.001 nM (e.g. 10⁸ M or less, e.g. from 10⁸ M to 10¹³ M, e.g., from 10⁹ M to 10¹³ M).

As used herein the term “predetermined cut-off value” means an amount of binding that is determined statistically to be indicative of a high likelihood of a disease (i.e. a cancer) or a particular severity thereof in a patient, in that a measured value of the target peptide in a patient sample that is at or above the statistical cut-off value corresponds to at least a 70% probability, preferably at least an 75% probability, more preferably at least an 80% probability, more preferably at least an 85% probability, more preferably at least a 90% probability, and most preferably at least a 95% probability of the presence of said disease.

As used herein, the term “values associated with normal healthy subjects” means standardised quantities of binding determined by the method described supra for samples from subjects considered to be healthy, i.e. without disease (i.e. a cancer); and the term “values associated with known disease severity” means standardised quantities of binding determined by the method described supra for samples from patients known to have disease (i.e. a cancer) of a known severity.

In a preferred embodiment, the cancer is bladder cancer, breast cancer, colorectal cancer, gastric cancer, head and neck cancer, lung cancer, ovarian cancer, pancreatic cancer, renal (kidney) cancer or melanoma. In another preferred embodiment, the cancer is a lung cancer and most preferably the lung cancer is non-small cell lung cancer.

In a preferred embodiment, the monoclonal antibody does not specifically bind to a peptide having the C-terminus amino acid sequence QRAFNKGPDPG (SEQ ID No. 2) (i.e. a version of the PRO-C9 target sequence extended at its C-terminus by the addition of a glycine residue). Preferably, the ratio of the affinity of said antibody for the PRO-C9 target sequence to the affinity of said antibody for the elongated version of the target sequence is at least 10 to 1, and more preferably is at least 20 to 1 or at least 30 to 1.

In a preferred embodiment, the monoclonal antibody does not specifically bind to a peptide having the C-terminus amino acid sequence QRAFNKGPD (SEQ ID No. 3) (i.e. a version of the PRO-C9 target sequence truncated by removal of the final proline residue). Preferably, the ratio of the affinity of said antibody for the PRO-C9 target sequence to the affinity of said antibody for the truncated version of the target sequence is at least 10 to 1, and more preferably is at least 20 to 1 or at least 30 to 1.

In a preferred embodiment, the monoclonal antibody is raised against a synthetic peptide having the C-terminus amino acid sequence QRAFNKGPDP. For example, the monoclonal antibody may be raised by: (a) immunizing a rodent (or other suitable mammal) with a synthetic peptide comprising the C-terminus sequence QRAFNKGPDP (SEQ ID. No:1), which peptide may optionally be linked at its N-terminus to an immunogenic carrier protein (such as keyhole limpet hemocyanin); (b) isolating and cloning a single antibody producing cell; and (c) assaying the resulting monoclonal antibodies to ensure that they have the desired specificity.

In a preferred embodiment the patient sample is selected from blood, serum or plasma.

In preferred embodiments the immunoassay is a competition assay or a sandwich assay. The immunoassay may, for example, be a radio-immunoassay or an enzyme-linked immunosorbent assay (ELISA). Such assays are techniques known to the person skilled in the art.

In a second aspect, the present invention provides a method of treating a cancer in a patient in need thereof, the method comprising:
(a) carrying out a method of immunoassay for detecting and/or monitoring a cancer in accordance with the first aspect of the present invention on a sample from a patient in order to detect whether the patient has a cancer; and
(b) administering to the patient a therapy for the treatment of said cancer if it is determined in step (a) that the patient has said cancer.

The therapy may be any therapy suitable for treating the cancer in question. The therapy may for example comprise or consist of one or more surgeries, one or more radiation therapies, one or more medicaments (such as for example one or more chemotherapies, one or more immunotherapies and/or one or more hormonal therapies), or combinations thereof. Medicaments may be formulated to topical or systemic administration. Topical medicaments may for example be formulated as creams, foams, gels, lotions, or ointments for administration. Systemic medicaments may for example be formulated for enteral or parenteral administration. Surgeries may be curative surgeries, preventative surgeries, debulking surgeries, palliative surgeries and/or restorative surgeries.

For example, where the cancer is lung cancer, suitable therapies may for example comprise one or more of: surgery, such as performing a lung resection such as for example a lobectomy, a sublobar excision (wedge resection) or removal of a whole lung (pneumonectomy); radiotherapy, examples of which include but are not limited to radiotherapy given together with chemotherapy, post-operative radiotherapy, brachytherapy (localized radiotherapy), prophylactic cranial irradiation, stereotactic radiation and palliative radiotherapy; chemotherapy using for example one more agents such as cisplatin, carboplatin, etoposide, gemcitabine, paclitaxel, docetaxel, vinorelbine, topotecan, irinotecan and pemetrexed; epidermal growth factor receptor (EGFR) inhibitor drugs such as erlotinib, gefitinib, afatinib, dacomitinib or osimertinib; targeted therapies using one or more drugs such as for example crizotinib and immunotherapy, using one or more monoclonal antibodies such as for example anti PD-Li monoclonal antibodies such as atezolizumab, nivolumab or pembrolizumab, monoclonal antibodies targeting cytotoxic T-lymphocyte-associated protein 4 (CTLA-4) such as ipilimumab, and/or monoclonal antibodies that target vascular endothelial growth factor such as bevacizumab, necitumumab, mobocertinib or cetuximab.

Where the lung cancer is non-small cell lung cancer, suitable therapies may for example comprise one or more of the lung cancer therapies described above.

In a third aspect, the present invention provides a monoclonal antibody that specifically binds to the C-terminus amino acid sequence QRAFNKGPDP (SEQ ID No. 1).

The antibody according to the third aspect of the invention is, in particular, suitable for use in carrying out the methods of immunoassay according to the first aspect of the invention. Preferred embodiments and features of the antibody according to the third aspect will therefore be apparent from the above discussion of the preferred embodiments of the methods according to the first aspect.

For example, in a preferred embodiment the monoclonal antibody does not specifically bind to a peptide having the C-terminus amino acid sequence QRAFNKGPDPG (SEQ ID No. 2); and/or does not specifically bind to a peptide having the C-terminus amino acid sequence QRAFNKGPD(SEQ ID No. 3); and/or is raised against a synthetic peptide having the C-terminus amino acid sequence QRAFNKGPDP(SEQ ID No. 1).

In a fourth aspect, the present invention provides an immunoassay kit comprising a monoclonal antibody in accordance with the third aspect of the present invention, and at least one of;
a streptavidin coated well plate;
a biotinylated peptide Biotin-L-QRAFNKGPDP (SEQ ID No. 18), wherein L is an optional linker;
a secondary antibody for use in a sandwich immunoassay;
a calibrator protein comprising the C-terminus amino acid sequence QRAFNKGPDP;
an antibody biotinylation kit;
an antibody HRP labelling kit;
an antibody radiolabelling kit; and
an assay visualisation kit.

The immunoassay kit according to the fourth aspect of the invention is, in particular, suitable for use in carrying out the method of immunoassay according to the first aspect of the invention. Further preferred embodiments and features of the immunoassay kit according to the fourth aspect will therefore be apparent from the above discussion of the preferred embodiments of the methods according to the first aspect.

### FIGURES

FIG. 1: Depiction of the three chains of type IX collagen (α1, α2 and α3) with the PRO-C9 target sequence on the α1 chain highlighted.

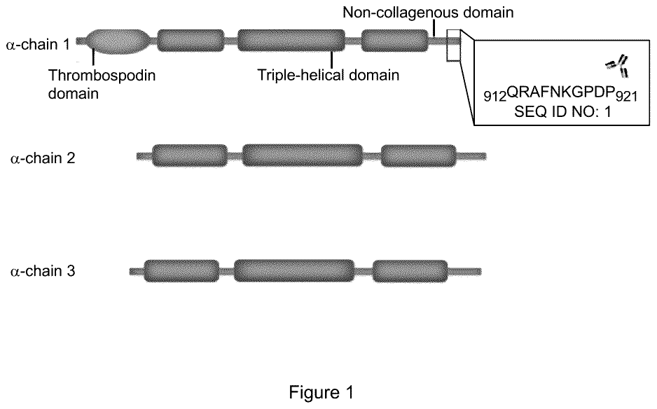

FIG. 2: Type IX collagen sequence alignment and specificity of the PRO-C9 assay. (A) Sequence alignment of the C-terminal region of the human, mouse and rat α1 chain of type IX collagen. The PRO-C9 sequence, and aligned C-terminus sequences of the mouse and rat α1 chains, are marked with a box. (B) Specificity of the PRO-C9 assay. Reactivity towards the standard peptide (QRAFNKGPDP SEQ ID No. 1), truncated peptide (QRAFNKGPD SEQ ID No. 3), elongated peptide (QRAFNKGPDPG SEQ ID No. 2) and non-sense standard peptide and coater (DQAAGGLRQH SEQ ID No. 14) are depicted. Signals are shown as relative luminescence (RLU) per second, as a function of standard peptide.

FIG. 3: Levels of PRO-C9 in healthy controls (n=43) and patients with NSCLC (n=40). (A) PRO-C9 is significantly elevated in patients with NSCLC compared to healthy controls (p=0.006). Data were analysed by t-test adjusted by age and gender and presented as Tukey Box-plots. (B) ROC Curve analysis was applied to evaluate the ability of PRO-C9 to distinguish between healthy controls and NSCLC patients.

FIG. 4: Levels of PRO-C9 in serum from cohort 1, including Healthy donors (n=13), bladder (n=19), breast (n=20), colorectal (n=20), gastric (n=20), head and neck (n=20), lung (n=20), pancreatic (n=20), prostate (n=20), or renal (n=20) cancer. Differences of PRO-C9 were evaluated by a Kruskal-Wallis test with Dunn's multiple comparison test, comparing the solid tumor types with healthy donors. ****p<0.0001, ***p<0.001, **p<0.0¹, *p<0.05.

### EXAMPLES

The presently disclosed embodiments are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

All reagents used were high quality chemicals from Merck (Whitehouse Station, NJ, USA) and Sigma (St. Louis MO, USA) unless stated otherwise. All synthetic peptides used for antibody production and assay validation were purchased from Genscript (Piscataway, NJ, US) (Table 1).

TABLE 1
Sequences of the synthetic peptides used for
monoclonal antibody production, assay
development and validation

| Peptide type | Sequence |
|---|---|
| Immunogenic peptide | KLH-CGG-QRAFNKGPDP |
| | (SEQ ID No. 15) |
| Selection peptide | QRAFNKGPDP |
| | (SEQ ID No. 1) |
| Elongated selection | QRAFNKGPDPG |
| peptide | (SEQ ID No. 2) |
| Truncated selection | QRAFNKGPD |
| peptide | (SEQ ID No. 3) |
| Non-sense coating and | DQAAGGLRQH |
| standard peptide | (SEQ ID No. 14) |
| Biotinylated selection | Biotin-QRAFNKGPDP |
| peptide | (SEQ ID No. 18) |

KHL = Keyhole Limpet Hemocyanin

### Monoclonal Antibody Development, Production, and Characterization

The amino acid sequence ⁹¹²‘QRAFNKGPDP’⁹²¹ was used for generation of monoclonal antibodies. Immunization was initiated by subcutaneous injection of 200 μl emulsified antigen and 100 μg immunogenic peptide (KLH-CGG-QRAFNKGPDP (SEQ ID No. 15)) in 4- to 6-week-old Balb/C mice using Stimmune (Thermo Fisher). The immunizations were repeated every second week until stable serum antibody titer levels were reached. The mouse with the highest serum titer was selected for fusion and rested for a month. Subsequently, the mouse was boosted intravenously with 50 μg immunogenic peptide in 100 μl 0.9% NaCl solution 3 days before isolation of the spleen for cell fusion. To produce hybridoma cells, the mouse spleen cells were fused with SP2/0 myeloma cells as described by Gefter et al. (10). Subsequently, the clones were plated into 96-well microtiter plates for further growth, and the limiting dilution method was applied to promote monoclonal growth.

An indirect ELISA performed on streptavidin-coated plates was used for the screening of supernatant reactivity. Biotin-QRAFNKGPDP was used as screening peptide, while the standard peptide QRAFNKGPDP was used to further test the specificity of the clones. Supernatant was collected from the hybridoma cells and purified using HiTrap affinity columns (GEHealthcare Life Science, Little Chalfront, Buckinghamshire, UK) according to manufacturer instructions and antibody isotype was determined using Rapid ELISA Mouse monoclonal antibody Isotyping Kit (Invitrogen, Carlsbad, CA, USA) following the manufacturer's protocol.

Native reactivity was assessed using human serum purchased from a commercial supplier (Valley Biomedical, Winchester, VA). The monoclonal antibody (mAb) was selected to specifically recognize the standard peptide (QRAFNKGPDP (SEQ ID No.1)), and not an elongated or truncated sequence of one amino acid (QRAFNKGPDPG (SEQ ID No. 2) and QRAFNKGPD (SEQ ID No.3), respectively).

The selected antibody was sequenced and the CDRs determined. The sequence of the chains are as follows (CDRs in bold; Framework sequence in Italics; Constant region underlined):

Light chain: Amino acid sequence (219 aa):
(SEQ ID No. 16)
NIMMTQSPSSLAVSAGEKATMSCKSSQSLLYSSNOMNYLAWYQQKPGQSP
KLLIYWASTRESDVPDRFTGSGSGTDFTLTISSVQTEDLAVYYCHQYFSS
RTFGGGTKLEIKRADAAPTVSIFPPSSEQLTSGGASVVCFLNNFYPKDIN
VKWKIDGSERQNGVLNSWTDQDSKDSTYSMSSTLTLTKDEYERHNSYTCE
ATHKTSTSPIVKSENRNEC
(SEQ ID No. 4)
CDR-L1: KSSQSLLYSSNOMNYLA
(SEQ ID No. 5)
CDR-L2: WASTRES
(SEQ ID No. 6)
CDR-L3: HOYFSSRT
Heavy chain: Amino acid sequence (448 aa):
(SEQ ID No. 17)
EVQLVESGGGLVQPKGSLKLSCAASGFTFNIYTMNWVRQAPGKGLEWVAR
IRSKSENYATYYADSVKDRFTISRDDSQSILYLQMNNLKTEDTAIYYCVR
QGIYYDYYGAMDYWGQGTSVTVSSAKTTPPSVYPLAPGSAAQTNSMVTLG
CLVKGYFPEPVTVTWNSGSLSSGVHTFPAVLQSDLYTLSSSVTVPSSTWP
SETVTCNVAHPASSTKVDKKIVPRDCGCKPCICTVPEVSSVFIFPPKPKD
VLTITLTPKVTCVVVDISKDDPEVQFSWFVDDVEVHTAQTQPREEQFNST
FRSVSELPIMHQDWLNGKEFKCRVNSAAFPAPIEKTISKTKGRPKAPQVY
TIPPPKEQMAKDKVSLTCMITDFFPEDITVEWQWNGQPAENYKNTQPIMD
TDGSYFVYSKLNVQKSNWEAGNTFTCSVLHEGLHNHHTEKSLSHSPGK
(SEQ ID NO. 7)
CDR-H1:IYTMN
(SEQ ID No. 8)
CDR-H2: RIRSKSENYATYYADSVKD
(SEQ ID No. 9)
CDR-H3: QGIYYDYYGAMDY

### PRO-C9 Assay Development

The development of the competitive chemiluminescence immunoassay (CLIA) included several preliminary optimizing experiments where reagents, concentrations, incubation-time and -temperature were analysed by several tests. The PRO-C9 competitive ELISA procedure was as follows: A 96-well streptavidin-coated white microplate (Greiner Bio-One, Kremsminster, Austria) was coated with 5 ng/mL biotinylated synthetic peptide (Biotin-QRAFNKGPDP SEQ ID No. 18) dissolved in assay buffer (10 mM phosphate buffered saline (PBS), 1% bovine serum albumin, 0.1% Tween-20, 0.36% Bronidox, 4 g/L NaCl, adjusted to pH 7.4 at 20° C.) and incubated for 30 min at 20° C. with constant shaking (300 rpm) in darkness.

Next, 20 μL/well of standard peptide (100 ng/mL) and samples were added to the appropriate wells, followed by the addition of 100 μL/well of HRP-labelled antibody diluted in assay buffer to the concertation of 100 ng/mL and incubated for 20 h at 4° C. with constant shaking (300 rpm) in darkness. After each incubation step, wells were washed with standard washing buffer (20 mM Tris, 50 mM NaCl, pH 7.2). The chemiluminescence substrate (Roche, BM Chemiluminescence ELISA substrate (POD), Basel, Switzerland) working solutions were mixed 15 min before use and 100 μL/well were added to plate and incubated for 3 min at 20° C. with constant shaking (300 rpm) in darkness. The relative light units were measured at all wavelengths within 5 min on a microplate luminometer reader (SpectraMax M5, Molecular Devices, CA, USA).

A standard curve was plotted using a 4-parameter logistic curve fit Y=(A−D)/(1+(x/C)\hat{ }B)+D, where R >0.9. Data were analysed using the SoftMax Pro version 7.0.3 software.

### Technical Evaluation

Two-fold dilutions of four human serum samples were used to assess linearity. Linearity was calculated as a percentage of recovery of the undiluted sample. Antibody specificity was calculated as percentage of signal inhibition by two-fold diluted standard peptide (QRAFNKGPDP (SEQ ID NO:1)), elongated peptide (QRAFNKGPDPG(SEQ ID NO:2)), truncated peptide (QRAFNKGPD (SEQ ID NO:3)), and non-sense peptide (DQAAGGLRQH (SEQ ID NO:14). The intra- and inter-assay variation was determined by 10 independent runs of five quality controls and two kit controls run in double determinations.

Accuracy of the assay was measured in healthy human serum samples spiked with standard peptide and a serum sample with a known high PRO-C9 concentration and calculated as the percentage recovery of the measured value and the expected concentration of the peptide or the serum sample with high PRO-C9 plus the concentration of the analyte in serum. Analytical interference was performed by adding a low/high content of haemoglobin (2.50/5 mg/mL), lipemia/lipids (1.50/5 mg/mL) and biotin (3/9 ng/mL) to a serum sample of known concentration. Recovery percentage was calculated with the normal serum sample as reference. The normal reference levels for haemoglobin, lipidaemia/lipids and biotin were 0-10 mg/dl (0-0.00161 mmol/L), <150 mg/dl (<1.6935 mmol/L) and 0.221-3.004 ng/ml, respectively. The interference was calculated as the percentage recovery of the analyte in non-spiked serum. The measurement range was defined as the range between lower limit of quantification range (LLOQ) and the upper limit of quantification range (ULOQ), which were determined from 10 independent runs with the standard peptide. Measurements below LLOQ or above ULOQ were assigned the value of LLOQ/ULOQ, respectively. IC50 (half-maximal inhibition concentration) was determined from the standard curve.

The analyte stability was examined through temperature tests and repeated freeze-thaw cycles of serum samples. The temperature tests included different time point and temperatures where PRO-C9 levels were measured in three human serum samples after 0, 2-, 4-, 24-, and 48-hours incubation at either 4° C. or 20° C. The recovery was estimated with the 0 hour samples as a reference. Furthermore, the effect of four repeated freeze/thaw cycles of three serum samples was assessed where freeze/thaw recovery was calculated with the zero cycle samples as a reference. Each sample were run in double determination.

### Biological Evaluation of PRO-C9

The biological utility of PRO-C9 was evaluated in serum samples patients with NSCLC (n=40) and healthy donors (n=43), from the commercial vendor Proteogenex (Culver City, CA). Serum samples were obtained and stored at −80° C. until use.

The biological utility of PRO-C9 was also evaluated in serum samples patients with various cancers (n=219) and healthy donors (n=13), from the commercial vendor Proteogenex (Culver City, CA). Serum samples were obtained and stored at −80° C. until use.

### Ethical Statement

All animals were treated according to the guidelines for animal welfare. Monoclonal antibody production in mice was approved by the Danish National Authority (The Animal Experiments Inspectorate) under approval number 2013-15-2934-00956. The collection and retrieval of the human cartilage complied with international ethical guidelines for handling human sample and patient information. All participants signed an informed consent, and the study was approved by the local ethical committee. Samples from both cohorts were collected after informed consent and approval by the local Ethical Committee and in compliance with the Helsinki Declaration of 1975.

### Statistical Analysis

PRO-C9 levels were log-transformed to obtain normality. Comparison of PRO-C9 between healthy controls and NSCLC was performed by a t-test corrected for age and gender. Diagnostic accuracy was tested by AUROC. A p-value below 0.05 was considered significant. Statistical analysis and graphs were performed using GraphPad Prism version 9 (GraphPad Software, Inc., La Jolla, CA) and R studio version 4.2.1 (R Foundation for Statistical Computing, Vienna, Austria. URL https://www.R-project.org).

### Results

### Technical Evaluation and Characterization of PRO-C9 Assay

The monoclonal antibody clone NBH313 #46 9H11-1D9-2B6 showed the best native reactivity, peptide affinity, and stability for the assay and was chosen for assay development. A summary of the technical evaluation of the PRO-C9 assay can be found in Table 2.

TABLE 2
Summary of technical parameters for PRO-C9 assay

| ASSAY PARAMETER | RESULT | |
|---|---|---|
| ELISA format | Competitive ELISA with | |
| | chemiluminescence | |
| Curve fit model | 4-point logistic curve fit | |
| Detection range: | 0.65-120 | (ng/mL) |
| LLOQ-ULOQ | | |
| LLOB | 0.41 | ng/ml |
| Mean Slope | 1.022 | |
| Mean IC25 | 1.20 | ng/mL |
| Mean IC50 | 3.62 | ng/mL |
| Mean IC75 | 10.61 | ng/mL |
| Intra-assay CV % | 2.35-5.93 | |
| Inter-assay CV % | 5.23-26.01 | |
| Dilution recovery (Range) | Accepted (94.6-100.8%) | |
| Accepted maximum freeze- | 5 freeze-thaw | |
| thaw (sample) | cycles (serum) | |
| Interference hemoglobin, | 94%/102% | |
| low/high | | |
| Interference lipid, | 103%/102% | |
| low/high | | |
| Interference biotin | >100 | ng/mL |

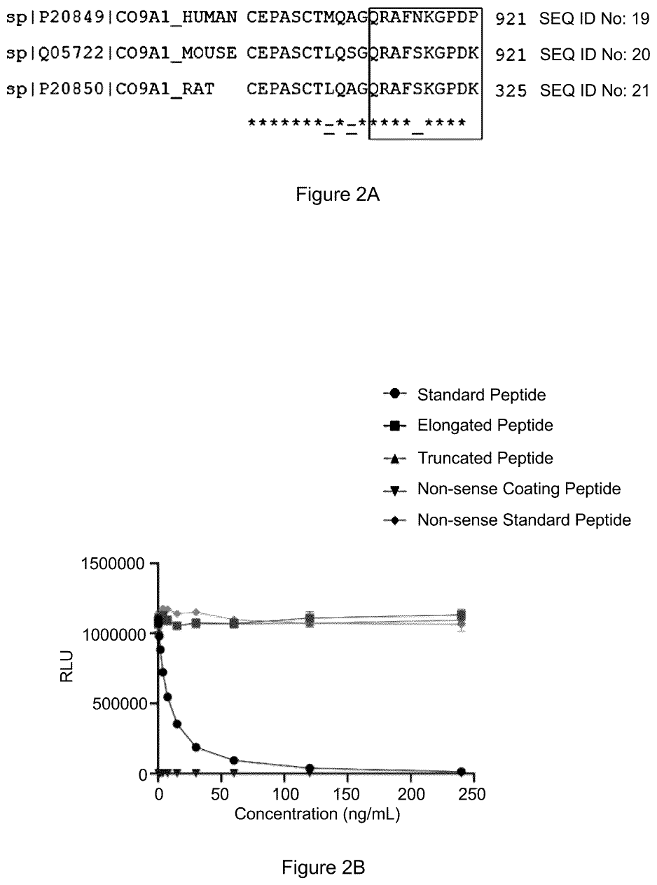

The measurement range (LLOQ-ULOQ) was determined to 0.65-120 ng/mL. The inter- and intra assay variation was 12.0% and 3.9% respectively, and linearity was approved from undiluted to a 2-fold dilution in human serum. The stability of the analyte was acceptable for five freeze-thaw cycles (92.1-112.8%). Haemoglobin, lipemia and biotin did not interfere with measurements of PRO-C9 in human serum. The human sequence was aligned using UNIPROT, and the corresponding sequence in mouse and rat had a mismatch on position 1 and 5 (FIG. 2A). To evaluate the specificity of the PRO-C9 assay, the mAb was tested towards the elongated peptide, truncated peptide, non-sense standard peptide and non-sense coater, and showed no reactivity towards those peptides (FIG. 2B).

### Baseline Demographic and Clinical Characteristics

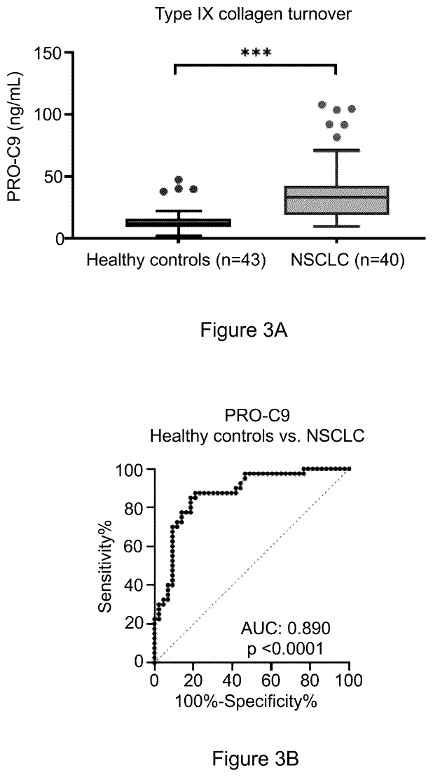

The PRO-C9 assay was measured in serum from patients with NSCLC. The patient cohort consisted of healthy controls and patients diagnosed with NSCLC. Patient demographics can be found in Table 3. Here patients with NSCLC had significantly higher levels of PRO-C9, compared to healthy controls when corrected for age and gender (p=0.006, FIG. 3A). The diagnostic power of PRO-C9 for a patient suffering from NSCLC compared to healthy controls was AUROC=0.890 (95% CI: 0.82-0.96 p<0.001, FIG. 3B).

TABLE 3
NSCLC Patient Demographics

| | Healthy | NSCLC | |
|---|---|---|---|
| | (N = 45) | (N = 40) | p value |
| Age | 43.3 (8.3) | 60.1 (7.0) | <0.01 |
| Sex | 22 (48.9%) | 31 (77.5%) | <0.01 |
| BMI | NA | 25.1 (3.4) | |
| Stage | | | |
| N-Miss | 45 | 0 | |
| IIIC | 0 | 4 (10.0%) | |
| IV | 0 | 3 (7.5%) | |
| IVA | 0 | 25 (62.5%) | |
| IVB | 0 | 7 (17.5%) | |
| lIIC | 0 | 1 (2.5%) | |
| Histological diagnosis | | | |
| adenocarcinoma | 0 | 26 (65.0%) | |
| squamous | 0 | 14 (35.0%) | |
| cell | | | |
| carcinoma | | | |
| PRO-C9 | 14.5 (9.5) | 39.7 (27.8) | <0.01 |

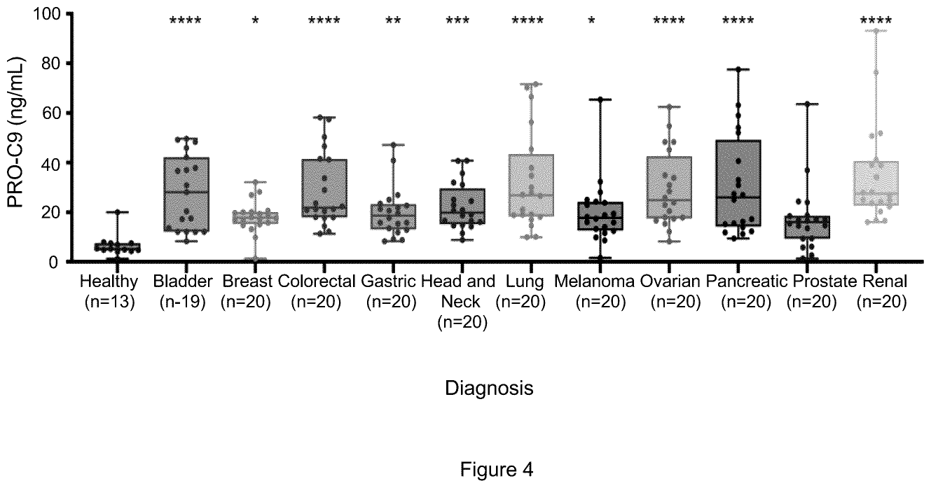

The PRO-C9 assay was used to analyse serum from second cohort of patients with a range of cancers. The second patient cohort consisted of healthy controls and patients diagnosed with cancer. Patient demographics can be found in Table 4. Here patients with bladder, breast, colorectal, gastric, head and neck, lung, ovarian, pancreatic and renal cancer as well as melanoma had significantly higher levels of PRO-C9, compared to healthy controls when corrected for age and gender (FIG. 4).

TABLE 4
Patient demographics of Cohort 2

| | cancer | healthy | |
|---|---|---|---|
| | (n = 219) | (N = 13) | p value |
| Sex, male | 129 (58.9%) | 7 (63%) | 0.735 |
| Age | 59.08 (10.65) | 47.15 (8.80) | 0.050 |
| Ethnicity | | | |
| Caucasian | 219 (100.0%) | 11 (100.0%) | 1.000 |
| Patient diagnosis | | | |
| bladder | 19 (8.7%) | | <0.0001 |
| breast | 20 (9.1%) | | <0.05 |
| CRC | 20 (9.1%) | | <0.0001 |
| Head and neck | 20 (9.1%) | | <0.001 |
| kidney | 20 (9.1%) | | <0.0001 |
| lung | 20 (9.1%) | | <0.0001 |
| melanoma | 20 (9.1%) | | <0.05 |
| ovarian | 20 (9.1%) | | <0.0001 |
| pancreatic | 20 (9.1%) | | <0.0001 |
| prostate | 20 (9.1%) | | |
| gastric | 20 (9.1%) | | <0.01 |

### DISCUSSION

The applicant has developed and characterized a competitive CLIA assay for detection of type IX collagen using a monoclonal antibody reactive with the PRO-C9 target sequence. The main findings were as follows: 1) the successful development of a technically robust and specific assay targeting a C-terminus sequence (PRO-C9) of the α-1 chain of type IX collagen; 2) PRO-C9 levels were measurable in human serum; 3) PRO-C9 levels were significantly elevated in patients with NSCLC, bladder, breast, colorectal, gastric, head and neck, lung, ovarian, pancreatic and renal cancers as well as melanoma compared to healthy controls; and 4) the PRO-C9 assay showed an AUROC=0.890 indicating its potential as a diagnostic biomarker.

This is the first demonstration that PRO-C9 can be measured non-invasively in blood and with biological relevance in patients with cancer including NSCLC.

In conclusion a CLIA targeting the PRO-C9 target sequence was developed and validated. PRO-C9 levels were quantified in serum from NSCLC and other cancer patients where the levels were significantly elevated compared to healthy controls and can serve as a cancer biomarker.

### REFERENCES

1. Bray F, Ferlay J, Soerjomataram I, Siegel R L, Torre L A, Jemal A. Global cancer statistics 2018: GLOBOCAN estimates of incidence and mortality worldwide for 36 cancers in 185 countries. C A Cancer J Clin Wiley; 2018; 68:394-424.
2. Molina J R, Yang P, Cassivi S D, Schild S E, Adjei A A. Non-small cell lung cancer: epidemiology, risk factors, treatment, and survivorship. Mayo Clin Proc [Internet] Mayo Clin Proc; 2008 [cited 2022 Oct. 21]; 83:584-94. Available from: https://pubmed.ncbi.nlm.nih.gov/18452692/
3. Nissen N I, Karsdal M, Willumsen N. Collagens and Cancer associated fibroblasts in the reactive stroma and its relation to Cancer biology. J Exp Clin Cancer Res [Internet]J Exp Clin Cancer Res; 2019 [cited 2022 Oct. 21]; 38. Available from: https://pubmed.ncbi.nlm.nih.gov/30841909/
4. Ricard-Blum S. The Collagen Family. Cold Spring Harb Perspect Biol 2011; 3:1-19.
5. Thorlacius-Ussing J, Manon-Jensen T, Sun S, Leeming D J, Sand J M, Karsdal M, et al. Serum Type XIX Collagen is Significantly Elevated in Non-Small Cell Lung Cancer: A Preliminary Study on Biomarker Potential. Cancers (Basel) [Internet] Cancers (Basel); 2020 [cited 2022 Oct. 21]; 12:1-14.

Available from: https://pubmed.ncbi.nlm.nih.gov/32527017/
6. Diab M. The role of type IX collagen in osteoarthritis and rheumatoid arthritis. Orthop Rev [Internet] Orthop Rev; 1993 [cited 2022 Oct. 21]; 22:165-70. Available from: https://pubmed.ncbi.nlm.nih.gov/8451069/
7. Parsons P, Gilbert S J, Vaughan-Thomas A, Sorrell D A, Notman R, Bishop M, et al. Type IX collagen interacts with fibronectin providing an important molecular bridge in articular cartilage. J Biol Chem [Internet]J Biol Chem; 2011 [cited 2022 Oct. 21]; 286:34986-97. Available from: https://pubmed.ncbi.nlm.nih.gov/21768108/
8. Chung C M, Lee C H, Chen M K, Lee K W, Lan C C E, Kwan A L, et al. Combined genetic biomarkers and betel quid chewing for identifying high-risk group for oral cancer occurrence. Cancer Prev Res [Internet] American Association for Cancer Research Inc.; 2017 [cited 2022 Oct. 21]; 10:355-61. Available from: https://aacrjournals.org/cancerpreventionresearch/article/10/6/3 55/258417/Combined-Genetic-Biomarkers-and-Betel-Quid-Chewing
9. Piotrowski A, Benetkiewicz M, Menzel U, Diaz De Stahl T, Mantripragada K, Grigelionis G, et al. Microarray-based survey of CpG islands identifies concurrent hyper- and hypomethylation patterns in tissues derived from patients with breast cancer. Genes Chromosomes Cancer [Internet] Genes Chromosomes Cancer; 2006 [cited 2022 Oct. 21]; 45:656-67. Available from: https://pubmed.ncbi.nlm.nih.gov/16575877/
10. Gefter M L, Margulies D H, Scharff M D. A simple method for polyethylene glycol-promoted hybridization of mouse myeloma cells. Somatic Cell Genet [Internet]1977; 3:231-6. Available from: http://www.ncbi.nlm.nih.gov/pubmed/605383

1: A method of immunoassay for detecting and/or monitoring a cancer in a patient, the method comprising;
i) contacting a patient sample with a monoclonal antibody that specifically binds to the C-terminus amino acid sequence QRAFNKGPDP (SEQ ID NO: 1);
ii) detecting and determining the amount of binding between said monoclonal antibody and peptides in the sample; and
iii) correlating said amount of binding with values associated with normal healthy subjects and/or values associated with known disease severity and/or values obtained from said patient at a previous time point and/or with a predetermined cut-off value.

2: The method of claim 1, wherein the cancer is bladder cancer, breast cancer, colorectal cancer, gastric cancer, head and neck cancer, lung cancer, ovarian cancer, pancreatic cancer, renal cancer or melanoma.

3: The method of claim 1, wherein the cancer is a lung cancer.

4: The method of claim 3, wherein the lung cancer is non-small cell lung cancer.

5: The method of claim 1, wherein the monoclonal antibody does not specifically bind to a peptide having the C-terminus amino acid sequence QRAFNKGPDPG (SEQ ID NO: 2).

6: The method of claim 1, wherein the monoclonal antibody does not specifically bind to a peptide having the C-terminus amino acid sequence QRAFNKGPD (SEQ ID NO: 3).

7: The method of claim 1, wherein the monoclonal antibody is raised against a synthetic peptide having the C-terminus amino acid sequence QRAFNKGPDP (SEQ ID NO: 1).

8: The method of claim 1, wherein the patient sample is blood, serum or plasma.

9: The method of claim 1, wherein the immunoassay is a competition assay or a sandwich assay.

10: The method of claim 1, wherein the immunoassay is a radio-immunoassay or an enzyme-linked immunosorbent assay.

11: A monoclonal antibody that specifically binds to the C-terminus amino acid sequence QRAFNKGPDP (SEQ ID NO: 1).

12: The monoclonal antibody of claim 11, wherein the monoclonal antibody does not specifically bind to a peptide having the C-terminus amino acid sequence QRAFNKGPDPG (SEQ ID NO: 2).

13: The monoclonal antibody of claim 11, wherein the monoclonal antibody does not specifically bind to a peptide having the C-terminus amino acid sequence QRAFNKGPD (SEQ ID NO: 3).

14: The monoclonal antibody n of claim 11, wherein the monoclonal antibody is raised against a synthetic peptide having the C-terminus amino acid sequence QRAFNKGPDP (SEQ ID NO: 1).

15: An immunoassay kit comprising a monoclonal antibody that specifically binds to the C-terminus amino acid sequence QRAFNKGPDP (SEQ ID NO: 1), and at least one of;
a streptavidin coated well plate;
a biotinylated peptide Biotin-L-QRAFNKGPDP, wherein L is an optional linker;
a secondary antibody for use in a sandwich immunoassay;
a calibrator protein comprising the N-terminus amino acid sequence QRAFNKGPDP (SEQ ID NO: 1);
an antibody biotinylation kit;
an antibody HRP labelling kit;
an antibody radiolabelling kit; or
an assay visualisation kit.

16: The immunoassay kit of claim 15, wherein the monoclonal antibody does not specifically bind to a peptide having the C-terminus amino acid sequence QRAFNKGPDPG (SEQ ID NO: 2).

17: The immunoassay kit of claim 15, wherein the monoclonal antibody does not specifically bind to a peptide having the C-terminus amino acid sequence QRAFNKGPD (SEQ ID NO: 3).

18: The immunoassay kit of claim 15, wherein the monoclonal antibody is raised against a synthetic peptide having the C-terminus amino acid sequence QRAFNKGPDP (SEQ ID NO: 1).


SEQUENCE LISTING (21 sequences)

SEQ ID NO: 1 - protein, 10 aa, Homo sapiens
QRAFNKGPDP

SEQ ID NO: 2 - protein, 11 aa, Homo sapiens
QRAFNKGPDPG

SEQ ID NO: 3 - protein, 9 aa, Homo sapiens
QRAFNKGPD

SEQ ID NO: 4 - protein, 17 aa, Mus musculus
KSSQSLLYSSNQMNYLA

SEQ ID NO: 5 - protein, 7 aa, Mus musculus
WASTRES

SEQ ID NO: 6 - protein, 8 aa, Mus musculus
HQYFSSRT

SEQ ID NO: 7 - protein, 5 aa, Mus musculus
IYTMN

SEQ ID NO: 8 - protein, 19 aa, Mus musculus
RIRSKSENYATYYADSVKD

SEQ ID NO: 9 - protein, 13 aa, Mus musculus
QGIYYDYYGAMDY

SEQ ID NO: 10 - protein, 79 aa, Mus musculus
KSSQSLLYSSNQMNYLAWYQQKPGQSPKLLIYWASTRESDVPDRFTGSGSGTDFTLTISSVQTEDLAVYYCHQYFSSRT

SEQ ID NO: 11 - protein, 83 aa, Mus musculus
IYTMNWVRQAPGKGLEWVARIRSKSENYATYYADSVKDRFTISRDDSQSILYLQMNNLKTEDTAIYYCVRQGIYYDYYGAMDY

SEQ ID NO: 12 - protein, 112 aa, Mus musculus
NIMMTQSPSSLAVSAGEKATMSCKSSQSLLYSSNQMNYLAWYQQKPGQSPKLLIYWASTRESDVPDRFTGSGSGTDFTLTISSVQTEDLAVYYCHQYFSSRTFGGGTKLEIK

SEQ ID NO: 13 - protein, 123 aa, Mus musculus
EVQLVESGGGLVQPKGSLKLSCAASGFTFNIYTMNWVRQAPGKGLEWVARIRSKSENYATYYADSVKDRFTISRDDSQSILYLQMNNLKTEDTAIYYCVRQGIYYDYYGAMDYWGQGTSVTVS

SEQ ID NO: 14 - protein, 10 aa, Homo sapiens
DQAAGGLRQH

SEQ ID NO: 15 - protein, 13 aa, synthetic construct
  MOD_RES 1: note = Keyhole Limpet Hemocyanin
CGGQRAFNKGPDP

SEQ ID NO: 16 - protein, 219 aa, Mus musculus
NIMMTQSPSSLAVSAGEKATMSCKSSQSLLYSSNQMNYLAWYQQKPGQSPKLLIYWASTRESDVPDRFTGSGSGTDFTLTISSVQTEDLAVYYCHQYFSSRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - protein, 448 aa, Mus musculus
EVQLVESGGGLVQPKGSLKLSCAASGFTFNIYTMNWVRQAPGKGLEWVARIRSKSENYATYYADSVKDRFTISRDDSQSILYLQMNNLKTEDTAIYYCVRQGIYYDYYGAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 18 - protein, 10 aa, synthetic construct
  MOD_RES 1: note = Biotinylated with optional linker
QRAFNKGPDP

SEQ ID NO: 19 - protein, 21 aa, Homo sapiens
CEPASCTMQAGQRAFNKGPDP

SEQ ID NO: 20 - protein, 21 aa, Mus musculus
CEPASCTLQSGQRAFNKGPDP

SEQ ID NO: 21 - protein, 21 aa, Rattus sp.
CEPASCTLQAGQRAFNKGPDP